(19) United States
(12) Patent Application Publication
(10) Pub. No.: US 2018/0070866 A1
(43) Pub. Date: Mar. 15, 2018

(54) NON-INVASIVE NANOSENSOR SYSTEM TO DETERMINE ANALYTE CONCENTRATION IN BLOOD AND/OR BODILY FLUIDS.

(71) Applicant: Kaamran Raahemifar, Waterloo (CA)

(72) Inventors: Kaamran Raahemifar, Kitchener-Waterloo (CA); Ehsan Marzbanrad, Kitchener-Waterloo (CA)

(21) Appl. No.: 15/264,010

(22) Filed: Sep. 13, 2016

(51) Int. Cl. A61B 5/1455; C12Q 1/00; G01N 33/66; G01N 33/49; A61B 5/145; A61B 5/1486; A61B 5/0205; A61B 5/00; A61B 5/0476

(52) U.S. Cl. CPC A61B 5/1455; C12Q 1/006; G01N 33/66; G01N 33/492; A61B 5/14503; A61B 5/1486; A61B 5/02438; A61B 5/02055; A61B 5/002; A61B 5/0022; A61B 5/0476; A61B 5/742; A61B 5/7465; A61B 5/14517

(57) ABSTRACT

The device is an ultra-low power, non-invasive in-vivo blood analyte sensor system incorporating multiple sensors including a carbon base and/or carbon base material coated with metallic nanoparticles and/or metallic nanoparticle nanoprobes, as a modified Clark electrode sensor system, that detects hydrogen peroxide concentrations, pH, and/or glucose concentrations (and other analytes) in bodily secretions (e.g. tears, saliva, sweat). The device consists of multiple chemoreceptive sensors, a microprocessor, a signal amplifier, signal filtering, error correction algorithms, analog-to-digital converter and wireless electromagnetic data transmitter to a remote device for further processing and/or data storage (e.g. on a server, on a cloud-based storage system, etc) and/or visual representation via software. The method involves applying the nanoprobe sensor array to skin tissue and the resulting electrical impulses correlate with glucose concentration within liquids such as tears, saliva, blood, etc. The collected data is then represented visually on a computer (handheld, smart-phone, desktop, laptop, etc) via software. The device is powered by ambient electromagnetic radiation, thermoelectric and/or solar power and/or rechargeable battery. The device is placed against the skin or immersed in a sample for sensor measurement. Single and continuous data collection is possible. The device can be reused repeatedly, re-sterilized and it is a high accuracy, low-cost option for multiple use glucose concentration measurements. The device can monitor blood glucose for Type I and Type II diabetics and it is suitable for a wide range of applications including gases, liquids and solids, biological, organic and inorganic chemical analysis.

### FIELD OF THE DISCLOSURE

The device is an ultra-low power, non-invasive in-vivo blood analyte sensor system incorporating multiple sensors including a carbon base and/or decorated carbon base material with metallic nanoparticles and/or metallic nanoparticle nanoprobes, as a modified Clark electrode sensor system, that detects hydrogen peroxide concentrations, pH, and/or glucose concentrations (and other analytes) in bodily secretions (e.g. tears, saliva, sweat). The device consists of multiple chemoreceptive sensors, a microprocessor, a signal amplifier, signal filtering, error correction algorithms, analog-to-digital converter and wireless electromagnetic data transmitter to a remote device for further processing and/or data storage (e.g. on a server, on a cloud-based storage system, etc) and/or visual representation via software. The method involves applying the nanoprobe sensor array to skin tissue and the resulting electrical impulses correlate with glucose concentration within liquids such as tears, saliva, blood, etc. The collected data is then represented visually on a computer (handheld, smart-phone, desktop, laptop, etc) via software. The device is powered by ambient electromagnetic radiation, thermoelectric and/or solar power and/or rechargeable battery. The device is placed against the skin or immersed in a sample for sensor measurement. Single and continuous data collection is possible. The device can be reused repeatedly, re-sterilized and it is a high accuracy, low-cost option for multiple use glucose concentration measurements. The device can monitor blood glucose for Type I and Type II diabetics and it is suitable for a wide range of applications including gases, liquids and solids, biological, organic and inorganic chemical analysis.

### BACKGROUND

1) Analyte Detection—E.g. Glucose

Glucose is a product of carbohydrate digestion and glycogen conversion via the liver. Glucose is a sugar and it is the primary source of energy for most cells. A narrow range of glucose concentration in the blood plasma is necessary for human survival. Glucose is one of many blood analytes that can be detected non-invasively using our technology and glucose detection represents a large commercial opportunity.

Glucose concentration in the body is regulated by insulin, glucagon, enzymes of the liver, thyroid and adrenal hormones. Glucose concentration is elevated in diabetes, liver disease, obesity, pancreatitis, due to steroid medications, or during stress. Low levels of glucose may lead to various conditions, namely: liver disease, overproduction of insulin, hypothyroidism, or alcoholism.

Insulin concentration informs the body of glucose concentration in blood plasma. Insulin originates in the pancreas. Insulin concentration in the blood increases following meals, since the digestion of food results in increased concentration of glucose in blood plasma. Insulin changes are relayed throughout the body, to the liver, muscles and fat cells. The role of insulin in the body, is to communicate to various organs, namely, to remove glucose from the blood and store the glucose in the form of fat or glycogen.

Insulin concentration and production is directly affected by aging of the human body and pancreatic disease or malfunction. Diabetes mellitus is a condition that is the result of the impairment of insulin production and low concentration of insulin in blood plasma. In this situation, unregulated glucose concentrations result in dangerously high glucose concentrations in the blood plasma. Elevated glucose concentrations result potentially fatal changes in the human body, namely, a) blood pH becomes acidic—as the body searches for alternative methods of delivering energy to cellular functions and; b) dehydration—the body attempts to remove excess sugar via urine.

### Measurement of Glucose Concentration—Overview:

methods of blood glucose analysis are more prevalent than non-invasive methods. For example, using a pin to draw blood from a fingertip and collect it for analysis and then placing that blood sample into a blood analysis device, commonly refereed to as a glucometer. The drawing of blood may be necessary up to 8 times per day, a process which can be inconvenient for diabetics. Recent technological advancements are shifting in favour of non-invasive methods, not requiring direct contact with blood plasma.

Non-invasive technologies include optical rotation, spectroscopic techniques, electrodes incorporating enzymes that react with a specific analyte in a bodily fluid (in this case glucose) measured on a handheld device. From these non-invasive methods, the concentration of blood glucose can be inferred with relative accuracy and precision. One particular non-invasive method that shows promise for longterm exploration, targets hydrogen peroxide concentrations in bodily fluids, such as tears, saliva, urine, to monitor blood glucose levels. Hydrogen peroxide concentration measurements are one of the targets for our sensor technology.

Measurement of Glucose Concentration—Invasive Methods (Direct Contact with Blood):

The most common example is pricking a finger with a needle. The blood flows out of the body by natural pressure differential between the human body and exterior environment and onto a thin metallic needle. The blood droplet adheres to the needle, where it is transfer manually to an external glucose concentration analysis device. The analysis device can be a variety of technologies. The simplest being a sheet of chemically treated paper whose colour changes relative to the blood glucose concentration. In an electronic sensor device, the blood can be analyzed by a sensor and/or chemical reaction in the device. The results of the sensor and/or chemical reaction generate a result, from which, a machine or individual can infer the blood glucose concentration. Either method of analysis yields a single analyzed result. Benefit: high accuracy and precision of results, via direct contact with blood. Drawback: painful, prolonged use of the procedure will damage skin, veins and capillaries, inconvenient, time consuming, continuous monitoring not possible.

One example is a skin mounted device with a needle indirect into the skin of the human. The blood flows out of the body by natural pressure differential between the human body and exterior environment and into a thin hollow metallic needle. The needle directs the blood to a collection unit. In the collection unit, the blood can be analyzed by a sensor and/or chemical reaction. The results of the sensor and/or chemical reaction generate a result, from which, a machine or individual can infer the blood glucose concentration. Benefit: high accuracy and precision of results, via direct contact with blood. Drawback: painful, prolonged use of the procedure will damage skin, veins and capillaries, inconvenient, time consuming, continuous monitoring not possible, possibility of infection. Another example is a skin mounted device with a device implant underneath the dermal layers of human skin, in direct contact with blood plasma. The blood flows around the sensor system with the body, where it interacts with a sensor or an electrode via electrochemical or enzyme catalyzed reaction. The information gathered from the sensor or electrode (in the form of electrons) is analyzed by a computer, where it will indicate the blood glucose concentration, continuous monitoring possible. Benefit: high accuracy and precision of results, via direct contact with blood. Drawback: painful, prolonged use of the procedure will damage skin, veins and capillaries, inconvenient, time consuming, surgery required for implantation and removal, possible rejection by immune system of host. Additional drawbacks for invasive glucose meters. The blood meters require a physical blood sample, which is a painful procedure and if repeatedly measured, thick calluses can form on the fingertips causing more pain over time to draw blood. Continuous glucose monitoring systems (CGMS) provide the ability to continuously monitor glucose levels, but they require additional calibration to blood samples. The monitors are also very costly. CGMS can cost several thousand dollars, and while blood monitors are relatively inexpensive, the electrodes are disposable and become costly over time. A single-use blood electrode strip costs about $1, and a CGMS 3-7 day sensor costs $30-$50. Therefore, a reusable, non-invasive glucose monitoring system would come in very handy and benefit all diabetic patients.

Measurement of Glucose Concentration—Non-Invasive Methods (No Contact with Blood):

These methods require the use of inference by means of using various types of surface-based electrodes of sensors. Sensor and electrode accuracy and precision, vary widely based upon its: a) design, b) material composition, and c) method of operation. Technological advancements in materials science and nanotechnology are shifting the focus of glucose concentration analysis in blood plasma, towards non-invasive technologies.

For non-invasive methods, sensors and electrodes measure blood analytes that are part of the larger, naturally occurring, biochemical process cycles that sustain the human body. The biochemical processes cycles include the production of, and regulation of, glucose concentration in blood plasma. Therefore, if one cannot access blood directly, one can access the chemical analytes that are products, or by-products, of the natural glucose biochemical cycle. The analytes can provide enough information, that would allow a device to infer, with a reasonable level of accuracy and precision, the concentration of glucose in blood plasma.

A nanosensor array sensitive to the presence of hydrogen peroxide in secreted bodily fluids (e.g. saliva, tears, sweat). Benefits include moderate to high accuracy, moderate to high precision, no discomfort, skin is not adversely affected by use, no contact with blood, continuous monitoring may be possible.

### 2) Sensor Technologies—Infrared Spectrography

This technology represents one of the sensor methods used in our device, to determine the concentration of an analyte non-invasively. The following steps are required to collect information regarding blood glucose concentrations in the blood plasma and body tissue, and to come up with a conclusion based upon the data.

Transmittance spectroscopy requires a near infrared light source that will be used on the skin. Near infrared light is transmitted from one side of the skin. Due to the presence of oxygen in the skin, the light experiences some loss in intensity, and thus is attenuated. A receiver sensor is placed on the opposite side of the skin. The receiver will detect the attenuated light transmitted through the skin. Assuming that glucose concentration in blood and the surrounding tissue is a constant, small concentration of blood would mean large transmittance. Conversely, a low transmittance would mean a large concentration of blood.

The spectral characteristics of water, protein, fat, urea, and glucose are all unique in the near-infrared from 1100 to 2500 nm. In previously published research, the following wavelengths bands were determined to provide useful information for glucose concentration analysis: a) 1111 nm to 1835 nm via nasal membrane and mucosa, b) 800 nm to 1300 nm via transmission through finger skin and tissue, c) 1429 nm to 2000 nm transmission through tongue tissue (Burmeister, 1998), (Blank, 1999), (Marbach, 1993), (Marbach 1995), (Jagemann, 1995), (Fischbacker, 1997), (Danzer, 1998) and (Muller, 1997). The methods use for obtaining non-invasive measurements were not practical for industrial, academic, medical, commercial or consumer markets, for regular testing. The common problem was found to be the weak glucose signal in the presence of skin (a multi layered structure) with widely varying heterogeneity of physiology, chemistry and/or structure. These variations generate dramatic and nonlinear changes in the optical properties of the tissue sample, even under highly controlled laboratory conditions. (Anderson, 1981), (Cheong, 1990), (Benaron, 1993), (Conway, 1994), (Homma, 1996), (Profio, 1989), (Van Gemert, 1989) and (Wilson, 1990). Therefore, scattering and scatter-correction were to be considered in any future technology for optical infrared glucose concentration analysis. Scattering of the infrared light transmission through skin and tissue can be accounted for, In mathematical error correction algorithms, by considering the following: 1) skin structure varies widely; 2) skin pigmentation (found in the basal layer) varies widely; and 3) skin consists of stratified layers, namely the cellular epidermis (10 to 150 micrometers thick, consisting of, basal, middle and superficial layers) and a dermis of connective tissue below the epidermis (Ebling, 1979). The superficial layer of skin is moist, however, the underlying layers increase in water content. The dermis layers of skin contain vasculature (blood vessels), with a very high concentration of blood for measurement purposes, ranging from 0.5 mm to 4 mm in diameter. The highest concentration of blood vessels with the smallest diameter were found on the face and eyelids, the larger blood vessel diameters were found on the back—the average diameter is 1.2 mm for the human body (Wilson, 1988). Therefore, an incident beam of infrared light directed perpendicular human skin (average thickness, consistency and vasculature) will attenuate by 93 to 96 percent of the incident beam intensity, due to absorption or scattering. Absorption and scattering collectively determine the penetration of light into skin, as well as reflectivity of scattered light from the skin.

Glucose concentration and water concentration in the human body is facilitated by the process of diffusion. Diffusion balances and distributes water and analytes in blood serum. Short-term intravascular and extravascular fluid balances are moderated by diffusion and Fick's Law. The absorption value of the light energy changes depending on the concentration of molecules in the absorbing material. Based on this principle, the intensity of light leaving the absorbing material, such as skin, can be used to determine the concentration of that particular material. Skin thickness and amount of blood determine whether the blood concentration inside the skin is at the required level.

skin tissue thickness is measured using the Beer-Lambert Law:

y=Ae−bx+C=−Dx+E Equation #1

x=optical power. y=penetration depth. A, b, C, D and E are absorption constants.

R(unscaled O2level)=(AC red/DC red)/(AC IR/DC IR) Equation #2.

This measurement procedure will provide data that will be analyzed using polynomial regression.

Regression analysis is used to identify the relationship between two or more quantitative variables. In this case, the analysis helps us quantify the relationship between measured voltage (independent variable X) and blood glucose concentration (dependent variable Y). The maximum and minimum voltage are measured. Based on these voltage values, glucose level is determined using polynomial regression. A 2nd order polynomial regression is used to determine the final equation used to interpret and calculate digital sensor data results.

### 3) Sensor Technologies—Modified Clark Sensors for Hydrogen Peroxide Detection

This technology represents another sensor method used in our device, to determine the concentration of an analyte non-invasively. The following steps are required to collect information regarding blood glucose concentrations in the blood plasma and body tissue, and to come up with a conclusion based upon the data.

One type of sensor, is one that is connected by a wire to a small pager-sized unit which records data and is later downloaded onto a computer. The tip of the sensor is made of a membrane selectively permeable to glucose. Once the glucose passes through the membrane, it is oxidized by the enzyme glucose oxidase. Reduced glucose oxidase is then oxidized by reacting with molecular oxygen, forming hydrogen peroxide as a by-product. At the electrode surface, hydrogen peroxide is oxidized into water, generating a current which can be measured and correlated to the glucose concentration outside the membrane. This type of device requires at least four finger sticks per day for calibration. Long term, this device is impractical and costly for individual or commercial use.

Another type of sensor, the one that is used in our device, is a Clark-type glucose biosensor that is modified to detect hydrogen peroxide. The sensor consists of platinum metal coated with glucose oxidase, an enzyme extracted from cows, and a semi-permeable membrane. The enzyme breaks down glucose forming hydrogen peroxide as a by-product. As the peroxide reacts with the metal it produces a current that is proportional to the concentration of glucose in the blood. The current is then converted into a glucose concentration. The vast majority of devices using this technology are invasive, however, some are non-invasive, analyzing bodily fluids secreted by the body to monitor glucose concentration. In our device, we have modified the Clark-type glucose biosensor for the purposes of non-invasive detection of concentrations of hydrogen peroxide (instead of measuring glucose directly, however, concentrations of glucose itself can be measured non-invasively as well in trace amount on the skin surface or in secreted bodily fluids). Hydrogen peroxide concentration measurements represent another critical chemoreceptive sensor in our device.

### Saliva Analysis:

A Saliva analyzing system is being developed that measures the saliva glucose level which has a somewhat direct correlation with blood glucose level. The iQuickit Saliva Analyzer was soliciting for public funding, but due to a lack of public support, the project terminated. A Tokyo University research developed a saliva analyzing system that uses an enzyme sensor by determining saliva glucose levels in the range of 0.1 to 1.0 mg/dl. Another study reported the correlation between saliva glucose and blood glucose for people with and without diabetes. It concluded that the correlation coefficient for people with diabetes (0.40) was so much worse than for those without (0.58) that it could be used as a potential screening method for diabetes, but neither of these values would allow glucose monitoring.

### Tear Drop Analysis:

There are many published technical articles that describe the relationship between glucose in eye tears and glucose in blood. While many claim that there are no significant correlation glucose content of blood and tears among normal and diabetic persons, there are some researches which show a possible connection. A company in Norway namely Noviosense has developed a sensor for glucose in tears that resides in the eyelid. Another publication from National Chiao Tung University proposed a contact lens which detects glucose level and sends data wirelessly. It is still a proposal and prototype has yet been developed. It depends on an electrochemical method based on oxidizing or reducing the target analytes and claims to achieve a real-time, quick-response, high-efficiency and cost-effective analysis. The enzyme based glucose sensor has the following electrochemical reaction: It starts from catalyzing glucose to hydrogen peroxide using the enzyme glucose oxidase (GOD). Hydrogen Peroxide is then oxidized at the electrode to release electrons, generating a current signal proportional to the glucose concentration.

After analyzed previous technologies used for tear non-invasive glucose detection, we have concluded that hydrogen peroxide concentrations were the most effective means available for non-invasive chemical analysis to determine blood glucose concentrations in blood plasma, in-vivo.

### 4) Sensor Fusion.

By means of combining data between two or more sensor inputs, and referencing the data against each other and reference levels, sensor fusion is an effective means of reducing errors and artifacts by emphasizing the accuracy and precision of the target data signals. For example, if two sensor methods are used, employing two different means of sensor detection (one using light, one using chemical detection), an error present in the light sensor will not be present in the chemical sensor—as they use different sensor methods to detect and generate signals. As a result, when the light data is crossreferences against the chemoreceptive sensor data, the target signal (e.g. glucose) will be detected in both sensors. Additional sensors are used (e.g. temperature) to calibrate the sensor data against a temperature specific correction factor, further improving accuracy. The error from the light signal can be subtracted from the overall data without adversely affective the quality of the combined data. In fact, the quality of the combined data will be enhanced noise, electronic filtering, software algorithms, neural network processing and machine learning will further improve the quality of data when referenced against regular sensor calibration data. Our device aims to be the most accurate analyte testing device available and our laboratory research to date, has demonstrated that this method is extremely effective when compared to the methods used by our competitors.

5) Market for Non-Invasive Blood Analyte Monitoring Systems (e.g. Glucose):

Diabetes is a leading cause of mortality among the world population. Diabetes is a chronic disease resulting in abnormal production and use of insulin. Insulin is a hormone produced naturally by the pancreas, facilitating glucose uptake into cells. Diabetics face an elevated risk of, or are affected by, one of the following conditions: heart disease and stroke, high blood pressure, kidney disease, neuropathy, retinopathy, skin conditions, gum disease, Impotence, and fetal complications. Diabetes currently afflicts 154 million people worldwide according to the World Health Organization: In 1997, approximately 60 million people diabetics were diagnosed among the G20 nations, with the number increasing to 300 million by the year 2025, at a rate of approximately eight hundred thousand new cases every year. In many cases, nearly 1 of 3 diabetics, they are not aware that they in fact suffering from diabetes. The estimated total cost to the United States economy alone exceeds $90 billion per year. Diabetes Statistics, National Institutes of Health, Publication No. 98-3926, Bethesda, Md. (November 1997).

There are two main factors for the increasing prevalence of diabetes. 1) Age. The prevalence of diabetes increases as the “baby boom” population demographic peak of the developed nations is shifting towards the age of 55 years and over, therefore, approximately 25% to 33% of the population of developed nations. With a rising ageing population, the prevalence of diabetes will increase proportionately. 2) Diet. The diet of an average person living in a developed country is exceeds the normal daily requirements for sugar intake by a factor of 2 to 5×. The resulting increased sugar concentration in blood places stress upon the neuroendocrine system, pancreas, and insulin production (to moderate the concentration of glucose in the blood), resulting in the early manifestation of diabetes. A demand for blood sugar concentration analysis exists within the global and developed world. In the past, blood sugar analysis was performed invasively, namely, the the drawing of blood from the skin, up to ten times per day. The blood droplet would be analyzed on a chemically treated paper medium, a table-top electronic device, or a liquid chemical assay for home use. The pain and inconvenience of this method was undesirable for diabetics. In the last 15 years, advances in technology have enabled noninvasive methods in blood glucose concentration analysis (for example, optical, impedance, ultrasound) have evolved. Presently, the miniaturization of the sensor devices, reduced costs of mass production and portability of new technologies make the process of living with diabetes, more comfortable. The demand for a non-invasive blood-sugar concentration monitoring device has evolved into a multibillion dollar industry, and our device provides a cost-effective, convenient, portable, non-invasive and novel solution, expanding upon previous generations of innovation in this field.

### 6) Assessment of Prior Art:

With respect to nanoprobe sensors, previously patented technologies demonstrate a need for an effective, accurate and precise, low-cost, reusable non-invasive glucose concentration measuring device, with limited success (U.S. Pat. No. 5,956,150, 1999), (U.S. Pat. No. 5,448,662, 1995), (U.S. Pat. No. 5,655,530, 1997), (U.S. Pat. No. 6,381,489, 2002), (2010-029399, Japan, 2008), (2009-108060, Huey-Fang, S, Taiwan, 2009), (2004-113434, Masato, N, Japan, 2004), (2011-245069, Masahide, O, Japan, 2011), (U.S. Pat. No. 7,640,140, Ruchti, T, 2009), (U.S. Pat. No. 7,787,924, Acosta, G, 2010), (U.S. Pat. No. 9,226,663, Fei, M, Canada, 2016), (20160157752, Cho, 5, USA, 2016), (WO/2016/035881, Katsuhiko, M, Japan, 2016). Some of the patented devices generated excessive artifacts, were sensitive to skin temperature variations, sensitive to skin moisture variations or were not practical for portable devices. Some of the devices targeted one area of the body exclusively (e.g. palm of hand) with inconsistencies, irradiate the entire body with infrared radiation with excessive scattering and incoherence due to skin surface variations, angle of reflection upon body parts (e.g. eye versus chest). Other devices require testing glucose by drawing blood samples, which is not within the scope of a non-invasive device.

A list of noted previous attempts for non-invasive analyte monitoring from sweat using carbon base or decorated carbon base material with metallic nanoparticles and/or metallic nanoparticle nanoprobe sensor systems include: 1) (10015557, 2005, WO, Andreas), (WO/2010/045247, 2010, WO, Potts), (WO/2010/045247, 2006, JP, Junichi), (2003315340, 2003, JP, Takao) and (U.S. Pat. No. 5,140,985, 1991, Schroeder). Sweat is less commonly used as a medium for analyte detection, however both research publications and previous patents have demonstrated limited success with reasonably accurate detection of hydrogen peroxide concentrations, to infer glucose concentration in blood. These devices are neither portable, self-powered, low-cost, reusable, nor light-weight.

A list of noted previous attempts for non-invasive analyte monitoring from saliva using carbon base or decorated carbon base material with metallic nanoparticles and/or metallic nanoparticle nanoprobe sensor systems include: 1) (U.S. Pat. No. 5,140,985, 1991, Schroeder), (Jayoung, 2014), (Hong Kong Polytechnic University, 2015), (Jurysta, 2009), (Falk, 2014), (Liao, 2015), (Lima-Aragão, 2016), (Liu, 2016), (Mascarenhas, 2014), (Mirzaii-Dizgah, 2013), (Mussavira, 2015), (Tremblay, 2012) and (Zhang, 2015) fail to demonstrate all of the following, in combination: 1) device portability; 2) implementation as a wearable device for short or long term monitoring; 3) powered or charged by ambient RF, solar, piezoelectric or thermoresistive energies; 4) evidence to support testing more than one material (e.g. glucose) at a time; 5) cost-effectiveness for mass production for individuals and consumers; 6) use of nanomaterials; 7) a light-weight device; 8) sensor shielding or alternative methods to further reduce ambient noise in a reasonable manner.

A device called GlucoWatch (Cygnus Inc, 2001) was introduced for commercial sale more than 15 years ago. It was a wristband that draws glucose towards the surface of skin where the glucose was detected by built-in sensors. At first, a small electric current was applied that caused sodium ions from the fluid between skin cells (which carry glucose) to migrate towards the device. The glucose then enters gel filled discs within the watch that contain an enzyme, glucose oxidase, which breaks down glucose into hydrogen peroxide and oxygen as mentioned in the previous paragraphs. The amount of hydrogen peroxide is then measured and acts as a proxy for glucose levels. This watch was later discontinued because users experienced discomfort from the electrical current.

Similarly, another device, a contact lens containing an analyte sensor was introduced for commercialization in 2015. It is portable, lightweight and powered by ambient electromagnetic radiation, however, there is insufficient evidence to support that it is capable of glucose analysis with sufficient accuracy or precision and the device is limited to a single-use for its analytical feature, and contact lenses have a short lifespan (8 to 24 hours), thereby increasing user costs and the device becomes a consumable product rather than a strategic low-cost health-care investment.

Based upon these previous attempts, research and patents, a market exists for a non-invasive biological and chemical analysis device, that is portable, light-weight, low-power devices, low-cost, with accurate and precise results and short testing times. The disclosure provides a versatile nanoprobe sensor system meeting these requirements.

### 7) Our Proposed Solution:

Our device uses a carbon base or decorated carbon base material with metallic nanoparticles and/or metallic nanoparticle nanoprobe sensor based upon promising research for nanotechnology, graphene and nanoparticles, oxidative enzymes, flexible circuitry, and effective signal amplification processes which are an emerging trend in sensor technology as demonstrated by the research of (Aliofkhazraei, 2016), (Zhang, 2015), (Zhu, 2016), (Caussen, 2011), (Lerner, 2013), (Liu, 2016) and (Zhang, 2015). Our device is a modification of a Clark-type sensor, using carbon base or decorated carbon base material with metallic nanoparticles and/or metallic nanoparticle nanosensors, in an array. The carbon base or decorated carbon base material with metallic nanoparticles and/or metallic nanoparticle nanosensor is coated with a chemical compound able to detect minute concentrations of hydrogen peroxide. Based upon the concentration of hydrogen peroxide in the bodily fluid (saliva, tears, sweat), a corresponding electrical current will be generated. The nanoprobe will be semi-isolated from the surrounding bodily fluid by means of a nanopore gel or membrane, therefore, the skin will never directly contact the nanoelectrode probe. The electrical current is then amplified in an ultra-low noise amplifier, filters (to remove noise and random spikes of current) via microcontroller. The microcontroller output is then transmitted wirelessly to a remote PC or computing device (e.g. desktop computer, smart phone, laptop, tablet, etc). At the remote PC, the data is processed further, filtered and visually represented to the end user. The nanoprobe sensor device can be powered by ambient RF, thermoelectric power generator, piezoelectric power generator and/or solar power.

Additional algorithms can be used to further enhance the quality of the collected data, such as machine learning or neural network algorithms. Machine learning processes correct error from the sensor data output, prior to providing a final result that can be stored and analyzed. Machine learning can use predictive methodology, to remove undesirable readings from the collected data, namely physiological artifacts temperature noise, humidity, and poor sensor contact with skin tissue. In addition, a Hogenauer (“CIC”) filter to low-pass filter can be applied to further isolate data from ambient noise. The nanoprobe software sensor system can discriminate between the energy arriving beyond the parameters of measurement, further reducing errors.

In addition, the analysis of gases, liquids and solid materials are possible with our technology (Rudolph-Mohr, 2013), (Randall, 2014), (Overton, 2015), (Janata, 1985), (Unisense, 2016), (Geladi, 1985), (Institute of Analytical Chemistry and Food Chemistry, 2016). This expands the market of our device into medicine, environmental monitoring, soil analysis, chemical analysis, microbial analysis and electromagnetic radiation analysis. The core technology for this device appears in slightly modified versions, in other patents, for specific applications.

### SUMMARY OF THE DISCLOSURE

In accordance with certain embodiments of the present disclosure, optical sensor arrangements suited for use in physiological and/or chemical and/or environmental monitoring devices that are used for medical, healthcare, rehabilitation, blood analyte monitoring, chemical analysis, physical training, exercise, and/or general health monitoring are disclosed. In some embodiments, the monitoring devices may be a wrist watch, bracelet, arm band, nose bridge device, wearable glasses, wearable adhesives, gloves, hats, helmets, headbands, t-shirts, undershirts, socks, underwear, and belts, comprising one or more optical sensor arrangements.

In accordance with certain embodiments of the present disclosure, each optical sensor arrangement may comprise one or more light sources and/or photo detectors. In such embodiments, the light sources may comprise two or more spaced apart light emitting diodes or laser. Each photo detector may be, for example, a photodiode. In some embodiments, each photodiode may be positioned near a corresponding light emitting diodes or laser, or between a corresponding pair of light emitting diodes or lasers.

### SUMMARY OF EMBODIMENTS

In various embodiments, the device, in its many possible user-defined configurations, is capable of use in any of the following environments, but not limited to the following, individually or in any combination: a) mining, construction, law enforcement, police, military application; b) drug analysis, biochemical analysis; c) microbial analysis; d) food, agriculture, livestock analysis; e) soil, air, water analysis; f) hospitals, physiotherapy, geriatric remote monitoring, patient monitoring, rehabilitation; gait correction; g) man-down alarm safety system; h) vehicle tracking, driver behaviour analysis, tire movement, tire corrective control, driverless vehicle technologies and/or; i) fitness, health, athletic monitoring, leisure activities, daily personal use.

In various embodiments, the device shall target one, and/or more, analytes within a specific sample.

In various embodiments, the device shall target one, and/or more, environmental conditions within a specific sample and/or the surrounding environment.

In various embodiments, the device shall target one, and/or more, biological features within a specific sample and/or the surrounding environment.

In various embodiments, the device shall target one, and/or more, chemicals within a specific sample and/or the surrounding environment.

In various embodiments, the device shall target one, and/or more, physiological features within a specific sample and/or the surrounding environment.

In various embodiments, the device shall incorporate one, and/or more, sensors such as accelerometer, gyroscope, magnetometer, pH, heart-rate monitor, humidity sensor, piezoelectric sensor, GPS sensor system, light sensor, audio sensor, vibration sensor, chemical sensors (such as, but not limited to, coal dust, carbon dioxide, carbon monoxide, nitric oxide, nitrous oxide, nitrate, hydrogen sulfide, oxygen, nitrogen, boron, sulfur, carbon, alcohols, aldehyes, ketones, organic compounds, explosives, weapons, controlled substances, etc.), radiological agents (beta, gammma, alpha radiation), electromagnetic radiation sensors (such as, but not limited to, biological materials sensors (such as, but not limited to, microbes, DNA, RNA, protein, carbohydrate, lipid, enzyme, virus, bacteria, prions, etc.).

In various embodiments, the device shall target one, and/or more, electromagnetic features within a specific sample and/or the surrounding environment, including, but not limited to, photographs, video and/or audio.

In various embodiments, the device shall target one, and/or more, radiological features within a specific sample and/or the surrounding environment, including, but not limited to, alpha, beta, gamma, X-ray, subatomic particles and/or cosmic ray particle detection.

In various embodiments, the device shall receive power wirelessly from the surrounding environment, by means of, but not limited to, electromagnetic radiation, radiological particles, heat and/or vibration.

In various embodiments, the device shall be a wearable device, such as, but not limited to, one that can be attached permanently and/or temporarily, to any part of the body, internally or externally.

In various embodiments, the device shall be a portable device, such as, but not limited to, one that can be attached permanently and/or temporarily, to any part of the body, internally or externally.

In various embodiments, the device shall be a fixed location device, such as, but not limited to, one that can be attached permanently and/or temporarily, to any part of the body, internally or externally, and/or any fixed object (e.g. table, chair, desk, wall, ceiling, floor, etc).

In various embodiments, the device shall encased in a material designed to isolate the electrical system of the device from the surrounding the environment. The encasement can consist of, but it is not limited to, plastic, graphene, boron-nitride, metal, wood, polymers, glass, nanomaterials, gels, meshes, etc.

In various embodiments, the device shall consist of a sensor (and/or nanoprobe sensors and/or an nanoprobe sensor array) and microcontroller system, the processed digital data. The data shall be forwarded to an output device such as, but not limited to, a wireless communication system and/or a visual display system integrated into the device.

In various embodiments, a device shall possess accompanying software to be installed on an external hardware device, such as, but not limited to, a personal computer, smart-phone, PDA, tablet, notebook, netbook, laptop, or other programmable wearable device, for the purposes of visualization and interpretation of sensor data.

In various embodiments, a device will possess a signal conversion system and/or signal filtering system and/or software to analyze sensor data and/or software to improve signal-to-noise ratio and/or software to remove artifacts from raw sensor data and/or sensor fusion processing methods, for the processing of digital and/or analog signals into a form to optimize efficiency within the microcontroller and central processing system.

In various embodiments, a device will possess a microcontroller system for the processing of digital and/or analog signals.

In various embodiments, a device will possess a wireless communications system, such as, but not limited to, electromagnetic communication protocols (e.g. 802.11a) and/or audio communication protocols and/or chemical communication protocols.

In various embodiments, a device may possess the ability to reference points within a sample and/or cross-reference a sample with a known material for comparison and data analysis.

In various embodiments, a device may possess a nanoprobe, or nanoprobes, which extend beyond the physical housing of the device and/or may be retractable within the device and/or may be removable from the device.

In various embodiments, a device may possess suitable contact with a sample (e.g. nanoprobe sensor making contact with human skin) and in said circumstances, the device may possess an ability to calibrate electronically and/or adjust physical distance from and/or within a sample.

In various embodiments, a device may be used for sensing, measuring, and display of physiological, chemical and environmental data gathered from a sample, including, but not limited to humans, animals, plants, microbes, situated in any indoor environment, outdoor environment, in any soil, liquid, solid, gel, gas or chemical medium, or as part of a larger multipurpose device for analytical purposes, not limited to biological sample analysis, oilfield liquid analysis, inorganic molecular analysis, organic molecular analysis, aerosol analysis, gas analysis, microbial analysis.

In various embodiments, a device may contain multiple nanoprobe sensors, consisting of a material demonstrating a high sensitivity to a specific analyte, with each nanoprobe sensor targeting a different analyte.

In various embodiments, a device may contain multiple nanoprobe sensors, consisting of a material demonstrating a high sensitivity to a specific analyte, with each nanoprobe sensor targeting the same analyte.

In various embodiments, the device may consist of a processor-based computing system representative of the type of computing system in which chemical and/or biological and/or environmental and/or biometric data is collected and analyzed.

In various embodiments, the device may include one or more hardware and/or software components configured to execute software programs, such as software for storing, processing, and analyzing data.

In various embodiments, the device circuit board may be in wired, wireless, or electrical contact with one another to facilitate the exchange of signals/information between them.

In various embodiments, the modules of the present receivers are present on integrated circuits.

In various embodiments, variations of the device may be defined as water resistant or waterproof upon meeting said criteria.

In various embodiments, the device may hermetically sealed.

In various embodiments, the device may incorporate multiple channels of measurement.

In various embodiments, the device may incorporate multiple data filtering methods and/or error correction methods.

In various embodiments, the device shall provide information about the following, in any combination, but is not limited to, body mass index, calories, SpO2 and other biometric information.

1. The core technology and features of the device are as follows. A device can be used individually and/or collectively. The device can be customized extensively by a user, meaning, that features and sensors that can be activated or deactivated by the user during a setup of the device prior to use, to perform concentration analysis measurements of any of the following: gases, liquids and solids, biological material, organic, inorganic chemicals, electromagnetic radiation and ionization radiation. The device consists of a physical shell that will allow the user to attach it to any body part. The device and/or its individual sensors may be attached to other body parts as required by the user, as a method to customize the device to the needs of the user. Each device contains any combination of the following: a) multiple chemoreceptive; b) electromagnetic and/or; c) environmental sensors. Each device contains a near-infrared wavelength pulse wave photodiode emitter source, a photoelectric sensor, multiple chemoreceptive, electromagnetic and environmental sensors, a near-infrared wavelength pulse wave photodiode emitter source, a photoelectric sensor, microprocessor, a signal amplifier, signal filtering, error correction algorithms, analog-to-digital converter and wireless electromagnetic data transmitter, to transmit processed data from a local device to a remote device for further processing and/or data storage (e.g. on a server, on a cloud-based storage system, etc) and/or visual representation via software. electrical system that is sufficiently isolated from the surrounding environment, in accordance with national and international, industrial, occupational health and safety standards (physical shell that will waterproof and/or water repellant and/or suitable for light underwater work, to a depth of X meters). The device provides data that can be streamed, recorded and stored locally and/or remotely, for the end-user, for the purposes of data science, such as correlative analysis, data collections, predictive modelling, artificial intelligence, data mining and machine learning.

2. The device, as indicated in claim #1, consists of non-invasive sensors and/or invasive sensors, and both are capable of single and/or continuous data collection.

3. The device, as indicated in claim #1, consists of sensors that requires physical contact with, in any combination: a) skin; b) objects; c) samples and/or; environmental mediums (e.g. air, water, etc).

4. The device, as indicated in claim #1, contains one or more modified Clark electrode chemoreceptive sensor systems, namely carbon base and/or decorated carbon-based materials with metallic nanoparticles and/or metallic nanoparticle nanoprobes.

5a. The device, as indicated in claim #4, requires physical contact with a liquid (for example, but not limited to, tears, saliva, sweat, etc.), to measure hydrogen peroxide concentrations and/or pH and/or glucose concentrations and/or other analytes in bodily secretions (for example, but not limited to, tears, saliva, sweat, etc.).

5b. The device, as indicated in claim #4, contains one or more near-infrared wavelength electromagnetic radiation pulse wave photodiode emitter source(s) and a photoelectric sensor(s).

5c. The device, as indicated in claim #4, uses near-infrared wavelength electromagnetic radiation transmission through an object (e.g. skin) and reflectivity data from the surface or inner contents of an object (e.g. skin) to determine the analyte concentrations in the fluid(s) underneath the surface of the object (e.g. skin).

6. The device, as indicated in claim #1, consists of a sensor system for detecting concentrations of analytes and/or particulate in any combination of the following: a) blood; b) bodily fluids (e.g. saliva, tears, sweat, etc); c) air; d) water; e) soil; f) gases; g) liquids, h) solids and/or i) pH.

7. The device, as indicated in claim #1, consists of multiple physical sensor types in any combination, including, but not limited to: a) accelerometer; b) gyroscope; c) magnetometer; d) vibration; e) temperature; f) humidity; g) heart rate activity measuring sensor; h) electroencephalography electrodes; i) one or more near-infrared wavelength electromagnetic radiation pulse wave photodiode emitter source(s) and a photoelectric sensor(s) and/or; j) near-infrared wavelength electromagnetic radiation transmission through an object (e.g. skin) and reflectivity data from the surface or inner contents of an object (e.g. skin) to determine the analyte concentrations in the fluid(s) underneath the surface of the object (e.g. skin).

8. The device, as indicated in claim #1, consists of a camera, capable of still photographs and/or video recording and/or video streaming and/or two-way communication.

9. The device, as indicated in claim #1, consists of a microphone, capable of audio recording and/or audio streaming and/or two-way communication.

10. The device, as indicated in claim #1, consists of at least two methods of wireless communication in any combination: a) short range communication (within 7 feet radius of a device transceiver); b) medium range communication (within 50 feet radius of a device transceiver); c) long-range communication (full geographic expanse of a computer network, user defined, from the device transceiver).

11a. The device, as indicated in claim #10a, consists of a wireless communication transceiver in the radio frequency electromagnetic spectrum, for example, but not limited to, Bluetooth, approximately 700 MHz range, for short-distance communication between an individual sensor modality mounted to a user and the local device and in order to specifically identify a single device. The communication range is limited to less than 7 feet from the device transceiver.

11b. The device, as indicated in claim #10a, consists of an encryption method, identification number and temporal identification encoding, to connect the Bluetooth signal physically attached to one user, to only communicate with the local device of the user, to minimize cross-talk between other users wearing similar devices and in order to specifically identify a single device, via a method different from that used in 10b and 10c above.

12a. The device, as indicated in claim #10b, consists of a wireless microwave communication transceiver, for example, but not limited to, ZigBee or Wifi, approximately 2.4 GHz range, for medium distance communication with a network access point. The communication range is limited to 50 feet radius from the device transceiver.

12b. The device, as indicated in claim #10b, consists of an encryption method, modulation and temporal identification encoding, to connect the wireless communication device physically attached to one user, to only communicate with the nearest network wireless node, to minimize cross-talk between other users wearing similar devices and in order to specifically identify a single device, via a method different from that used in 10a and 10c above.

13. The device, as indicated in claim #10c, consists of an encryption method and identification number, to convey data from the wireless access node in a fixed location and/or ad-hoc computer network to communication directly with a remote computer and/or remote device (including, but not limited to, a handheld computer, smart-phone, desktop, tablet, netbook, laptop, etc), where the collected data is represented visually via software, in order to minimize cross-talk between other users wearing similar devices and in order to specifically identify a single device, via a method different from that used in 10a and 10b above. The remote device and/or remote computer shall be capable of accurately locating the individuals using RF based localization techniques.

14. The device, as indicated in claim #1, uses near-infrared wavelength electromagnetic radiation transmission through an object (e.g. skin) and reflectivity data from the surface or inner contents of an object (e.g. skin) to determine the analyte concentrations in the fluid(s) underneath the surface of the object (e.g. skin).

15. The device, as indicated in claim #1, contains error correcting coding and/or neural networks and/or machine learning and/or software algorithms and/or electronic filtering methods to generate processed data with high accuracy and high precision.

16. The device, as indicated in claim #1, is a device with ultra-low power requirements for operation, for energy efficiency, powered by ambient thermoelectric and/or electromagnetic radiation and/or piezoelectric generator and/or rechargeable batteries and/or rechargeable supercapacitors.
